(19) United States
(12) Patent Application Publication
(10) Pub. No.: US 2016/0184340 A1
(43) Pub. Date: Jun. 30, 2016

(54) THE USE OF SPINOSYNS AND SPINOSYN COMPOSITIONS AS LOCAL ANESTHETICS AND AS ANTIARRHYTHMIC AGENTS

(71) Applicants: Christine KRITIKOU, Kifissia (GR); Entarco SA, Kifissiagr (GR)

(72) Inventor: Christine KRITIKOU, (US)

(21) Appl. No.: 13/996,820

(22) PCT Filed: Dec. 21, 2011
(86) PCT No.: PCT/IB11/03309

§ 371 Date: Aug. 5, 2013

Related U.S. Application Data
(60) Provisional application No. 61/426,386, filed on Dec. 22, 2010.

(51) Int. Cl. A61K 31/7048; A61K 45/06; A61K 9/00

(52) U.S. Cl. CPC A61K 31/7048; A61K 9/0019; A61K 9/0014; A61K 45/06

(57) ABSTRACT

The present invention relates to the use of spinosyns and spinosyn compositions as local anesthetics and antiarrhythmic agents. Advantageously, spinosyns may be used as local anesthetics and/or antiarrhythmic agents with little or no disruption or harm to the host which may be an animal or human.

The present invention relates to the use of spinosyns and spinosyn compositions as local anesthetics and antiarrhythmic agents. Advantageously, spinosyns may be used as local anesthetics and/or antiarrhythmic agents with little or no disruption or harm to the host which may be an animal or human.

Local anesthetics are drugs that can produce a reversible loss of sensation, when applied to nerve tissues and for a limited region of the body, while maintaining consciousness (they do so by reversibly blocking the nerve conductances that transmit the feeling of pain from a specific locus to the brain). They are used as the backbone ingredients for local and regional anesthetic techniques, in the treatment of acute pain and for analgesia in the operative and post operative period. They are also used in the management of chronic pain where local anesthetic injections may have a prolonged effect, and are used to aid diagnosis and management prior to neurolytic procedures. Sodium channel blockade is a common feature of class I antiarrhythmic drugs and local anesthetics (Jann-Inn Tzeng et al. “The Cutaneous Analgesic Effect of Class I Antiarrhythmic Drugs”, International Anesthesia Research Society, Vol. 104, No. 4, April 2007). A variety of drugs, such as lidocaine and mexiletine, have both antiarrhythmic and local anesthetic properties, therefore in addition to the well recognized analgesic nerve blocking functions, some local anesthetic agents also have a role in the treatment of cardiac arrhythmias (e.g. lidocaine), as mucosal vasoconstrictors (e.g. cocaine) etc. Local anesthetics contrast with analgesics (painkillers) which relieve pain without eliminating sensation. Furthermore local anesthetics do not interact with the pain receptors or inhibit the release or the biosynthesis of pain mediators. When an analgesic agent is not capable of inducing the desired topical loss of pain, a local anesthetic may be recommended to bring local anesthesia and consequently analgesia.

Impulses are conducted along nerves by the movement of sodium, potassium and calcium ions across the nerve membrane during a rapid event called an action potential. The altered distribution of these ions briefly reverses the electrical polarity of the membrane for 1-2 ms, generating small local electrical currents that are propagated along the nerve as a wave. In addition to passive and active ion channels the membrane contains voltage-gated sodium channels, which open or close depending on the membrane potential difference. Local anesthetics interfere with the conduction process of the nerve tissues by blocking use-dependent voltage-gated Na⁺ channels. If a sufficient number of sodium channels are blocked the threshold potential will not be reached and impulse conduction stops. The resting membrane and threshold potential remain the same, but the action potential is temporarily halted. The degree of neuronal block is affected by the diameter of the nerve. Large diameter fibers (touch/pressure/motor) require higher concentrations of local anesthetic to achieve a given degree of block, compared with small myelinated fibers (pain afferents). As the block proceeds different sensory modalities are lost in the order of pain, temperature touch, deep pressure then motor function. At present, more than ten types of local anesthetics are in use, including, for example, bupivacaine, lidocaine, cocaine, mepivacaine, tetracaine, procaine, amethocaine, prilocaine, levobupivacaine, dibucaine and ropivacaine. These drugs can be cataloged into esters and amides according to their metabolic processes, in which the former type is metabolized mainly in the blood through hydrolysis by esterases whereas the latter type is metabolized in the liver. In terms of pharmacological mechanism, these two types of local anesthetics both achieve effects of infiltrative cutaneous anesthesia, peripheral nerve blocking, and spinal/epidural anesthesia through Na⁺ channel blocking (McLure, et al. (2005), Minerva Anesthesiol, 71:59-74 (2005).

Local anesthetics, when applied locally to a nerve tissue in appropriate concentrations, reversibly block the action potentials responsible for nerve conduction. Local anesthetics act on any part of the nervous system and on every type of nerve fiber. Thus, a local anesthetic in contact with a nerve trunk may cause both sensory and motor paralysis in the area enervated. Therefore, although the goal of topical or regional anesthesia is to block the transmission of signals in nociceptors to prevent pain, the administration of local anesthetics also produces numbness from block of low-threshold pressure and touch receptors, paralysis from block of motor axons, and block of autonomic fibers. A strategy for generating pain-restricted local anesthesia while preserving motor and autonomic responses is desirable in conditions such as childbirth, some dental procedures or in treating nociceptor-driven chronic pain such as postherpetic neuralgia. Often we desire a prolonged anesthetic effect but do not wish to use high concentrations of common local anesthetics or the repetitive administration of such anesthetics as this may lead to undesirable side effects. It should be noted that although a number of current clinically useful local anesthetic agents have been introduced into the market, an ideal local anesthetic drug has, unfortunately, not been realized. Existing local anesthetic formulations are limited by the short duration of their cutaneous analgesic effects and their exhibited toxicity when used at higher concentrations. A longer lasting local anesthetic with minimal side effects and toxicity, would thus be desirable.

Therefore according to one embodiment of the invention, is provided a novel method for producing local anesthesia, preferably with long duration of action, by using a spinosyn containing composition. According to another embodiment of the invention is provided a method of maximum local anesthesia with minimum risk of toxicity. According to yet another embodiment of the present invention is provided topical anesthetic formulations that can be used to provide relief from pain over a period of time. It is believed, although not wishing to be bound by theory, that an advantage of the spinosyns of the present invention, is their ability to retain their activity by exhibiting a much slower rate of metabolism in comparison with other known anesthetics, like for example lidocaine. In addition (and again, not wishing to be bound by theory), the extra size introduced by spinosyn molecule, with a molecular weight around 740—in contrast to the known anesthetics eg. lidocaine or derivative structures thereof—reduce the ability of the spinosyns to cross the blood-brain barrier, thus minimizing the risk of CNS toxicity.

Specialized excitable tissue in the heart initiates and conducts the electrical impulse that spreads through the myocardium and drives the cycle of contraction and relaxation. This process is mediated by voltage-gated sodium channels. Antiarrhythmic agents are a group of pharmaceuticals that are used to suppress abnormal rhythms of the heart (cardiac arrhythmias), such as atrial fibrillation, atrial flutter, ventricular tachycardia and ventricular fibrillation. The antiarrhythmic agents may act in various ways, depending on their molecular structure, e.g., by interfering with the sodium Na⁺ channel, by being anti-sympathetic nervous system agents and/or beta-blockers, by affecting potassium (K⁺) channels, by affecting the Ca²⁺ channels or by working by other or unknown mechanism.

In cardiomyocytes, the rapid component of the delayed rectifier K⁺ current, Iₖᵣ is an important repolarizing potassium current. Iₖᵣ is encoded by the human ether-a-go-go-related gene (hERG). This has been demonstrated in macroscopic current measurements and single channel measurements. HERG channels are one primary target for the pharmacological management of arrhythmias. Many class III antiarrhythmic drugs prolong the cardiac action potential and thereby the refractory period by blocking Iₖᵣ. Likewise, block of hERG channels by an agent contributes to its antiarrhythmic properties. Moderate hERG blockade may produce a beneficial antiarrhythmic effect. Since human ether-a-go-go (hERG) K⁺ channels play a critical role in cardiac arrhythmias, the effect of spinosyns and spinosad in particular on hERG K⁺ channels was investigated.

The present invention is based on the primary discovery that spinosyn, and spinosad in particular, has potent local anesthetic properties, based on the in vivo testing in rats. Without wishing to be bound by theory, it is believed that spinosyn acts by reversibly blocking the action potentials responsible for nerve conduction. Moreover the present invention is based on the discovery that spinosyns inhibit hERG potassium channels and thus, are potent antiarrhythmic agents. More specifically, according to one embodiment, it is an object of this invention to provide a local anesthetic and/or an antiarrhythmic composition comprising a pharmaceutical carrier and a spinosyn or spinosyn derivative or salt thereof, along with a method for reversibly blocking the action potentials responsible for nerve conduction and a method of treating and/or preventing cardiac arrhythmias.

Spinosyns have been commercially developed by Dow AgroSciences and have heretofore been used most commonly as pesticides/insecticides and to treat ectoparasites. This group of macrolides, originally discovered by Eli Lilly scientists in the search for new pharmaceuticals, has never been proposed for use as a local anesthetic and/or as an antiarrhythmic agent. Further, the prior work on these compounds (safety evaluation of spinosad insecticide K. E. Stebbins, D. M. Bond, M. N. Novilla and M. J. Reasor, “Spinosad insecticide: Subchronic and chronic Toxicity and Lack of Carcinogenicity in CD-1 Mice” Toxicological Sciences 65:276-287 2002) states that Spinosad has no known pharmacological activity in mice (with reference to an unpublished report to Horii, D.). In addition, the prevalent suggestion for the mode of action of spinosyns is, their action via nicotinic acetylcholine receptors nAChRs in insects causing involuntary muscle contractions and subsequent death. This suggestion does not teach nor presume neither a local anesthetic effect nor an antiarrhythmic effect. Moreover, the chemical structure of spinosyns do not fall within a local anesthetic basic structure properties; thus, the Structure-Activity-Relationship (SAR) rules are not applied in this case (Foye's principles of medicinal Chemistry, 6^{th} edition, Thomas L. Lemke et al, pages 471-473). This drug category (inhibitors of nerve conduction: local anesthetics), could be now further extended so as to include a wider list of molecules. Therefore, the present results are quite unexpected and the present inventor has not only recognized new properties and applications of spinosyns, but has shed light on a wider medical area, where heretofore innovation was only linked to modifications and substitutions on already- and since longtime-existing molecules. With spinosyns being added in this drug group, the whole category should be considered and studied from another perspective.

Other uses for spinosyns have been reported. As described in Published U.S. Patent Application 2007/0167379, incorporated herein by reference, spinosyns may be used in humans and mammals to promote or accelerate wound healing in both normal and healing impaired cases. According to this published application, Spinosad stimulates the neurogenic activation of healing, and subsequent inflammatory activity involved in cell growth and proliferation (it stimulates epithelial cell proliferation and basal keratinocytes for the purpose of wound healing). The same patent further proposes that the effect of spinosad on vascular flow and further to wound healing is mediated through the sensory nerve peptides substance P and CGRP and through endothelial nitric oxide. None of the above proposals could lead the skilled in the art in suspecting a local anesthetic property; quite the contrary, all these suggestions seem to be misleading for the present application, as they all teach a stimulation of local afferent sensory nerves rather than blocking nerve conduction in sensory and/or motor neurons, as it is the case with a local anesthetic molecule. In addition, many publications teach us that some local anesthetics inhibit would healing (“Local anesthetics and wound healing” Milos Chvapil M. D., Ph. D. et al, University of Arizona, Department of Anesthesiology, Journal of Surgical Research, Volume 27, Issue 6, December 1979, pages 367-371) or that impair wound healing (“Adverse effects of local anesthetic infiltration on wound healing” Brower M C et al., Reg. Anesth. Pain Med 2003; 28: 233-40). Based on the above, the finding that spinosyns and spinosad in particular has local anesthetic properties was quite unexpected.

Based on its insecticidal features, spinosad (as well as any other insect repellent/insecticide), has been also proposed to be used with a local anesthetic agent and a detectable marker, in the preparation of a topical anesthetic composition, as it is stated in U.S. Patent Application 2008/0085245. Spinosad is used (example 6) as an insecticide agent, together with the local anesthetics lignocaine and bupivacaine in the preparation of a local anesthetic composition in combination with a detectable marker. In this case spinosad is being used as an insect repellent, to stop insects from infesting a wound of an animal without suspecting or recognizing spinosad's anesthetic effect.

Other uses of spinosyns known in the art include, single-dose spinosyn oral veterinary formulations used for controlling an ectoparasite infestation on a companion animal for a prolonged time. The advantages of these oral systemic treatments are the killing of the ectoparasites (fleas), by ingestion of animals' blood that contains spinosyn, in contrast to contact killing by topical applications. In addition, spinosad cream rinse 0.9% has proven to be very effective for head lice treatment in children (Stough D., Pediatrics. 2009; 124:e389-e395).

Other uses of spinosyns are described in PCT International Application No. PCT/IB2010/001713 entitled “The Use Of Spinosyns And Spinosyn Compositions Against Diseases Caused By Protozoans, Viral Infections And Cancer,” filed on Jun. 23, 2010, and U.S. Provisional Application No. 61/220,059 filed Jun. 24, 2009, both of which are incorporated herein by reference in their entirety.

According to one embodiment of the present invention, spinosyns, more specifically spinosad, can be used as a local anesthetic agent, preferably with a prolonged duration of action. According to yet another embodiment of the present invention, spinosyns, more specifically spinosad, can be used as an antiarrhythmic agent.

According to another embodiment, the present invention relates to pharmaceutical local anesthetic compositions, including veterinary compositions, comprising at least one spinosyn or derivative or salt thereof. According to one embodiment, the at least one spinosyn is the main local anesthetic agent. According to another embodiment, the spinosyn is the only local anesthetic agent. Compositions according to this embodiment of the invention contain at least one spinosyn in an amount effective to have an anesthetic effect and which may not be harmful to a host, for example, a human or an animal.

In one embodiment, the present invention relates to a method of inducing a local anesthetic effect and/or analgesic effect and prolonged loss of pain sensation, the method comprising administering a spinosyn composition according to the invention to the host in need thereof, i.e., the human or animal. The above may be used for temporary pain or chronic pain conditions (e.g., myofascial pain, muscle pain, back pain, osteoarthritis, osteomyelitis, osteosarcoma, neuropathic pain, postherpetic neuralgia, pruritus), as well as for preventing pain in procedures such as for example venipuncture or hair removal or topical surgical operation, dental operation or other medical operation, local infiltration or, use as a spinal/epidural (intrathecal) anesthesia agent. Spinal (intrathecal) block technique involves the injection of a local anesthetic into the cerebrospinal fluid at the lower part of the backbone, thereby temporarily stopping the nerve impulse transmission and the sensation of pain from a part of the body (spinal anesthesia). The method of inducing a long-term local pain relief may also be used for a wide variety of conditions in humans, including but not limited to: open reduction of fractures with internal fixation; reductions of fractures generally; injection of therapeutic substances into joints or ligaments; removal of implanted devices from bone; bunionectomy; treatment of toe deformities generally; knee arthroscopy; arthroscopy generally; division of joint capsule ligament, or cartilage; excision of semilunar cartilage of knee; synovectomy; other incision and excision of joint structure; total hip replacement; total knee replacement; repair of knee generally; repair of joints generally; excision of lesion of muscle, tendon, fascia, and bursa; other operations generally on muscles, tendons, fascia, and bursa; amputation of upper limb; amputation of lower limb; and other operations generally on the musculoskeletal system.

In yet another embodiment, the present invention relates to a method of combining general anesthesia with a spinosyn local anesthetic formulation for surgical pain relief or perioperative analgesia.

According to another aspect of the present invention, there is provided a method for providing prolonged analgesia to a subject in need thereof, said method comprising the step of applying topically at a site, a composition comprising at least one spinosyn or spinosyn derivative or salt thereof as a local anesthetic agent and a carrier, wherein the composition provides an anesthetic and consequently analgesic effect, for a period of at least 48 hours or 24 hours or 12 hours or 8 hours or 4 hours or 2 hours. When we inject slow release or sustained release spinosyn formulations for local pain relief then, the formulation demonstrates performance of full sensory response returning in 3-5 days or 8-10 days or even 20 days.

In another embodiment, the present invention relates to the use of spinosyn topical anesthetic formulations specifically on mucous membranes, such as in the eyes or mouth. Topical anesthesia is more rapid and efficacious when used on such regions as the structure of skin allows easy penetration and direct access to nerve fibers.

According yet to another embodiment, the present invention relates to the use of spinosyns or spinosyn derivatives or salt thereof, in pharmaceutical antiarrhythmic compositions, including veterinary compositions. Compositions according to this embodiment of the invention contain at least one spinosyn in an amount effective to have an antiarrhythmic effect and which may not be harmful to a host, for example, a human or an animal.

In another embodiment, the present invention relates to a method of treating or preventing cardiac arrhythmias, e.g., atrial fibrillation, atrial flutter, atrial arrhythmia and supraventricular tachycardia in a host, in need of an antiarrhythmic agent, which comprises administering to said host, i.e., the human or animal an effective amount of a spinosyn composition according to the present invention.

Another embodiment, of the present invention relates to a method of treating tachycardia in a host which comprises treating the patient with an antitachycardia device (e.g., a defibrillator or a pacemaker) in combination with an effective amount of a spinosyn composition.

The present invention also includes methods and compositions useful in facilitating spinosyn delivery for inducing the local anesthetic and/or antiarrhythmic effect, in the human or animal body.

In a particular embodiment, the present invention relates to a pharmaceutical composition comprising at least one spinosyn or derivative or salt thereof and a suitable carrier, for use as a local anesthetic and/or antiarrhythmic agent.

As used herein, “effective to induce an anesthetic effect,” or “local anesthetic effective amount” or “antiarrhythmic effective amount” or “pharmaceutically effective amount” refers to an amount of spinosyn capable of inducing, a local anesthetic effect or an antiarrhythmic effect within the host at a reasonable benefit/risk ratio applicable to any medical treatment. Further as used herein, the terms “effective,” “effective amount,” “effective in the control of,” and “effective for control” or “control” are all used interchangeably and all refer to the ability of the composition/active to act as a local anesthetic and/or antiarrhythmic agent compared to a non-active containing composition. As will be appreciated by those of ordinary skill in this art, the effective amount of a drug may vary depending on such factors as the desired biological endpoint, the drug to be delivered, the inclusion of any additional active or inactive ingredients, the target tissue, the route of administration, the duration of action, etc.

The terms “treat,” “treated,” or “treating,” when used with respect to administration to a host, refer to a therapeutic regimen that provides a local anesthetic effect and/or prevents, reduces or eliminates the tachycardia and arrhythmia in a host. The term “treatment,” as used herein, refers to the act of treating, as “treating” is defined above.

A “host” as used in the present invention, refers to humans and animals. The term animal includes all animals. Examples of animals are non-ruminants and ruminants. Ruminant animals include, for example, animals such as sheep, goat, and cattle, e.g., cows including beef cattle and dairy cows. According to one embodiment, the animal is a non-ruminant animal. Non-ruminant animals include household pets, e.g., dogs or cats as well as mono-gastric animals, e.g., pig or swine (including, but not limited to, piglets, growing pigs, and sows); poultry including turkeys, ducks and chickens (including but not limited to broiler chicks, layers); fish (including but not limited to salmon, trout, tilapia, catfish and carp); and crustaceans (including but not limited to shrimp and prawn).

As used herein, the term “active agent” or “therapeutic agent” means any compound or composition, which, upon being administered to a host, is capable of being a benefit in inducing a desired pharmaceutical effect.

As used herein, the term “facilitating delivery” or “to facilitate delivery” of a therapeutic agent to a host cell, means enhancing the uptake of a therapeutic agent in a cell to a level higher than the level of uptake of the therapeutic agent in an otherwise identical host cell which is not administered a compound or composition of the invention. The uptake of a therapeutic agent can be enhanced, by way of example and not by limitation, by any one or more of the following means: by by-passing the requirement for a cellular active transport mechanism for uptake of the therapeutic agent into a cell; by providing the therapeutic agent (i.e., a drug) intracellularly in an activated form, thereby bypassing the requirement for intracellular activation of the therapeutic agent by an enzyme such as an intracellular kinase; by overcoming a physiological barrier to uptake of the therapeutic agent in a desired cell, such as low solubility, poor absorption from the stomach or small intestine, or impermeability to the blood-brain barrier, and by enabling delivery of the therapeutic agent to sites not normally accessible thereto (i.e., CNS and lymphoid tissues).

As used herein a “biological membrane” is any membrane made of cells or tissues that serves as a barrier to at least some foreign entities or otherwise undesirable materials. As used herein a “biological membrane” includes those membranes that are associated with physiological protective barriers including, for example: the blood-brain barrier (BBB); the blood-cerebrospinal fluid barrier; the blood-placental barrier; the blood-milk barrier; and mucosal barriers including the vaginal mucosa, urethral mucosa, anal mucosa, buccal mucosa, sublingual mucosa and rectal mucosa. Unless the context clearly dictates otherwise, the term “biological membrane” does not include those membranes associated with the middle gastro-intestinal tract (e.g., stomach and small intestines).

A “biological membrane crossing rate,” provides a measure of a compound's ability to cross a biological membrane, such as the membrane associated with the blood-brain barrier (“BBB”). A variety of methods may be used to assess transport of a molecule across any given biological membrane. Methods to assess the biological membrane crossing rate associated with any given biological barrier (e.g., the blood-cerebrospinal fluid barrier, the blood-placental barrier, the blood-milk barrier, the intestinal barrier, and so forth), are known, described in the relevant literature, and/or may be determined by one of ordinary skill in the art.

The spinosyn of the invention may be used (i) in therapy, e.g., for the treatment of arrhythmia or temporary pain or chronic pain, (ii) prevention, e.g., treatment to prevent the onset of arrhythmia, and/or the recurrence of symptoms in an existing arrhythmia or to prevent the pain sensation prior to a pain causing treatment or operation.

The spinosyn compositions of the invention may be used (a) in veterinary medicine, which is the application of medical, diagnostic, and therapeutic principles to companion, domestic, exotic, wildlife, and production animals; and/or (b) in human medicine.

Spinosyns are known fermentation products derived from the naturally occurring bacteria Saccharopolyspora spinosa. The family of compounds derived from this bacteria are generally known as spinosyns and have been referred to as factors or components A, B, C, D, E, F, G, H, I, J, K, L, M, N, O, P, Q, R, S, T, U, V, W, Y, and the like, as described in U.S. Pat. Nos. 5,362,634, and 6,821,526 and published applications WO 93/09126 and WO 94/20518, which are each incorporated herein by reference in their entirety. The spinosyn compounds consist of a 5,6,5-tricyclic ring system, fused to a 12-membered macrocyclic lactone, a neutral sugar (rhamnose), and an amino sugar (forosamine) (see Kirst et al. “Unique Fermentation-derived Tetracyclic macrolides, Tetrahedon Letters, A83543A-D, 32:4839-4842, (1991)). As used herein, the term “spinosyn” refers to a class of compounds which are based upon the fermentation products from the naturally occurring bacteria, Saccharopolyspora spinosa and Saccharopolyspora pogona (species and subspecies and mutants thereof) or a biologically modified form of these bacteria or combinations thereof. Natural spinosyn compounds may be produced via fermentation from cultures deposited as NRRL 18719, 18537, 18538, 18539, 18743, 18395, and 18823 of the stock culture collection of the Midwest Area Northern Regional Research Center, Agricultural Research Service, United States Department of Agriculture, 1815 North University Street, Peoria, III. 61604. Spinosyn compounds are also disclosed in U.S. Pat. Nos. 5,496,931, 5,670,364, 5,591,606, 5,571,901, 5,202,242, 5,767,253, 5,840,861, 5,670,486 and 5,631,155. As used herein, the term “spinosyn” is intended to include natural factors and semi-synthetic derivatives of the naturally produced factors. A large number of chemical modifications to these spinosyn compounds have been made, sometimes referred to as spinosoids and are disclosed in U.S. Pat. No. 6,001,981, hereby incorporated by reference. The term “spinosyn” also includes the novel biologically-active compounds as described in published U.S. Patent Application No. 2006/0040877 produced by methods of using the hybrid polyketide synthase DNA to change the products made by spinosyn producing strains. Finally, the term “spinosyn” includes new spinosyn derivatives produced using the cloned Saccharopolyspora spinosa DNA as described in U.S. Pat. No. 7,015,001. Different patterns of control may be provided by biosynthetic intermediates of the spinosyns or by their derivatives produced in vivo, or by derivatives resulting from their chemical modification in vitro. Such biosynthetic (derived biologically) or synthetic (derived chemically) or semi-synthetic (derived biologically and then modified chemically) intermediates of the spinosyns are considered to belong to the class of “spinosyns” as described herein for use in the present invention.

Macrolide insecticides related to the spinosyns have been isolated from Saccharopolyspora sp. LW107129 (NRRL 30141 and mutants thereof). These compounds are disclosed in U.S. Pat. No. 6,800,614, herein incorporated by reference. These butenyl-spinosyn compounds—also called pogonins from the Saccharopolyspora pogona sp. differ from the known spinosyns with reference to the group attached at C-21 of the macrolide (i.e., 1-butenyl,1-propenyl etc) and optionally have new groups linked with the oxygen at C-17 of the macrolide (“Butenyl-spinosyns, a natural example of genetic engineering of antibiotic biosynthetic genes”. Journal of Industrial Microbiology & Biotechnology, Vol. 33, no 2, pp. 94-104, February 2006). A group of these spinosyns have a new 14-carbon macrolide ring system. Natural and semi-synthetic derivatives of the 21-butenyl and related spinosyns are also disclosed in U.S. Pat. No. 6,919,464, herein incorporated by reference. These compounds, are prepared directly or indirectly by modifying the compounds naturally produced by LW107129 or mutants thereof, which contain inactivated O-methyltransferase genes. They are structurally similar to the “classical” spinosyns, therefore the name spinosyn has been kept for these compounds as well. The three main structural elements in these molecules in which variations were seen are: (i) the macrocyclic ring system, (ii) the sugar attached to C-17 and (iii) the side chain attached to C-21, therefore the new naming system (nomenclature) is a composite of these three elements and as an example, we refer to spinosyn α1, spinosyn β1, spinosyn δ1, spinosyn α4, spinosyn β3, spinosyn β4, spinosyn α1a, spinosyn β1a, etc. (“Discovery of the butenyl-spinosyn insecticides: Novel macrolides from the new bacterial strain Saccharopolyspora pogona” P. Lewer at al, Dow Agrosciences, Bioorganic & Medicinal Chemistry 17 (2009) 4185-4196). Accordingly, the term spinosyn as used herein, is intended to include all of the above natural factors and semi-synthetic and synthetic derivatives of the naturally produced factors or combinations thereof.

Spinosyns and derivatives thereof can also exist in the form of pharmaceutically-acceptable salts and all crystalline forms of such salts. The term “pharmaceutically-acceptable salt” includes those salts that are, within the scope of sound medical judgment, suitable for use in contact with the tissues of humans and animals without undue toxicity, irritation, and allergic response, and are commensurate with a reasonable benefit/risk ratio. Pharmaceutically-acceptable salts are well known in the art. The salts may be prepared in-situ during the final isolation and purification of the compounds of the invention or separately by reacting a free base function with a suitable acid. By way of non-limiting example, spinosyns can form salts with hydrochloric, hydrobromic, sulfuric, phosphoric, acetic, benzoic, citric, malonic, salicylic, fumaric, oxalic, succinic, tartaric, lactic, gluconic, ascorbic, maleic, aspartic, cholic, glutamic, phthalic, picric, cinnamic, sorbic, benzenesulfonic, methanesulfonic, ethanesulfonic, hydroxymethanesulfonic, and hydroxyethanesulfonic acids. Additionally, by way of non-limiting example, pharmaceutically acceptable basic addition salts include cations based on alkali metals or alkaline earth metals such as lithium, sodium, potassium, calcium, magnesium, and aluminum salts, and nontoxic quaternary ammonia and amine cations including ammonium, tetramethylammonium, tetraethylammonium, methylamine, dimethylamine, trimethylamine, triethylamine, diethylamine, and ethylamine. Other representative organic amines useful for the formation of base addition salts include ethylenediamine, ethanolamine, diethanolamine, piperidine, and piperazine.

The term spinosyn also includes all isomers of the compounds, including constitutional (structural) isomers and stereoisomers (spatial). The stereoisomers include diastereomers and enantiomers. The diastereomers include cis-trans isomers, anomers, conformers and rotamers. The term spinosyn also includes racemic mixtures, optically active mixtures and combinations thereof.

The spinosyns of the present invention can be also in the form of a hydrate, which means that the compound further includes a stoichiometric or non-stoichiometric amount of water bound by non-covalent intermolecular forces, in the form of a solvate or a conjugate and salts thereof. “Solvate” means a physical association of a compound of the invention with one or more solvent molecules. This physical association involves varying degrees of ionic and covalent bonding, including hydrogen bonding.

In addition the term “spinosyn” as used herein, refers also to spinosyns produced by any fungal strains capable of producing spinosyn, e.g., fungal strains belonging to the genus Aspergillus, as mentioned in Patent Application WO/2009/054003, herein incorporated by reference.

The terms “derivative” or “structural analog” or “analog” or “homolog” all refer to a compound that is derived from a similar compound if one or more atoms, functional groups, or substructures have been replaced with different atoms, groups, or substructures. The term also refers to compounds that at least theoretically can be formed from the precursor compound.

Spinosad is an insecticide produced by Dow AgroSciences LLC (Indianapolis, Ind.) that is comprised of approximately 85% spinosyn A and approximately 15% spinosyn D. Spinosad is an active ingredient in several insecticide formulations available commercially from Dow AgroSciences LLC, including, for example, those marketed under the trade names TRACER®, SUCCESS®, SPINTOR®, LASER®, and ENTRUST®. The TRACER® product, for example, is comprised of about 44% to about 48% Spinosad (w/v), while ENTRUST® is a white to off-white solid powder containing about 80% Spinosad.

Spinosad, is also commercially available from the company Sigma-Aldrich for R&D purposes, as an analytical standard, at a purity of approximately 98% and is comprised mainly of approximately 70% spinosyn A and 30% spinosyn D.

Spinetoram is a semi-synthetic spinosyn, available commercially from Dow AgroSciences LLC in several insecticide formulations, including, for example, those marketed under the trade names DELEGATE® and RADIANT®. Spinetoram is the common name for a mixture of 50-90% (2R,3αR,5αR,5βS,9S,13S,14R,16αS,16βR)-2-(6-deoxy-3-O-ethyl-2,4-di-O-methyl-α-L-mannopyranosyloxy)-13-[(2R,5S,6R)-5-(dimethylamino)tetrahydro-6-methylpyran-2-yloxy]-9-ethyl-2,3,3α,4,5,5α, 5β,6,9,10,11,12,13,14,16α,6β-hexadecahydro-14-methyl-1H-as-indaceno[3,2-d]oxacyclododecine-7,15-dione, and 50-10% (2R,3αR,5αS,5βS,9S,13S,14R,16αS,16βS)-2-(6-deoxy-3-O-ethyl-2,4-di-O-methyl-α-L-mannopyranosyloxy)-13-[(2R,5S,6R)-5-(dimethylamino)tetrahydro-6-methylpyran-2-yloxy]-9-ethyl-2,3,3α,5α,5β,6,9,10,11,12,13,14,16α,16β-tetradecahydro-4,14-dimethyl-1H-as-indaceno[3,2-d]oxacyclododecine-7,15-dione. Synthesis of the components of spinetoram is described in U.S. Pat. No. 6,001,981. Spinetoram causes persistent activation of nicotinic acetylcholine receptors via an allosteric mechanism in the insect central nervous system. This particular mode of action is unique to spinetoram and the related active ingredient spinosad. The two compounds are almost structurally identical (Spinetoram (XDE-175) evaluation report ERC2008-01 Health Canada Pest Management Regulatory Agency). The extension of the 3′-O-alkyl moiety from methyl (spinosyn A) to its 3′-O-ethyl derivative increased the lipophilic nature of spinetoram while the hydrogenation of the 5,6-double bond improved its UV stability relative to spinosyn A (T. C. Sparks et al. “Neural network-based QSAR and insecticide discovery: spinetoram,” Springer Science+Business media B.V. 2008).

Spinosad, a safe and environmentally-friendly pesticide, derived from the fermentation juices of a soil bacterium called Saccharopolyspora spinosa, has been granted organic status by the USDA National Organic Program (NOP) in 2003 and Dow Agrosciences LLC, main producer of spinosyns, was presented by the U.S Environmental Protection Agency, with the Presidential Green Chemistry Challenge Award in the past for spinosad and in 2008 for spinetoram as well, as both products adhere to the principles of green chemistry. To chemists, spinosad is a complex molecule known as a “glycosylated macrolactone.” It acts against pests as a stomach and contact poison with a unique and not well understood heretofore mode of action. The present invention, has for the first time linked spinosyn and more specifically spinosad with local anesthetic activity and antiarrhythmic activity.

While not wishing to be bound by theory regarding spinosyns' mechanism of action in this application, it is believed that spinosyns present in the local anesthetic and antiarrhrythmic compositions of this invention, function by reversibly disrupting the coordinated sequence of ion movements (e.g., Na⁺, K⁺, Ca⁺⁺), in which sodium initially enters the cell, followed by a calcium influx, and finally a potassium efflux returns the cell to its resting state. Spinosyns, and spinosad in particular, alters these ion fluxes required for the initiation and conduction of impulses and thus a) results in reversibly blocking nerve conductance (i.e., inducing an anesthetic effect) and b) suppressing cardiac electrical activity of abnormally polarized cardiac tissues, thus decreasing myocardial contractility and reducing blood pressure. Again, and while not wishing to be bound by theory, the present inventor believes that spinosyns, like most of the clinically used local anesthetics which have a lipophilic portion and are tertiary amines with a pKa of 6.5-9.0, they first penetrate the nerve membrane in their un-ionized forms. Thus, under physiological conditions, both protonated forms and the un-ionized molecular forms are available for binding to the channel proteins. In fact, the ratio between the ions [BH]⁺ and the un-ionized molecules [B], can be easily calculated based on the pH of the medium and the pKa of the drug molecule by the Henderson-Hasselbach equation: pH=pKa−log [BH]⁺/[B]. For this reason, structural modifications that alter the lipid solubility, pKa and metabolic inactivation have a pronounced effect on the ability of a spinosyn molecule to reach or bind to the hypothetical receptor sites, thus modifying its local anesthetic properties. Thus, prerequisite for a spinosyn activity is its pKa to be in the range of 6.5-8.5 and to have a considerable lipophilicity, i.e. log P between 2.5-6.5.

The terms inhibitory concentration IC₅₀ and IC₁₀₀ (or alternatively lethal dose LD₅₀ and LD₁₀₀) refer to the concentration of the active that results in 50% and 100% cell death respectively. Typically, the IC₅₀ and IC₁₀₀ may be determined in vitro. Growth, and subsequent inhibition of growth, is determined by methods also well known in the art. Alternatively, the IC₅₀ and IC₁₀₀ may be determined in vivo. Growth, and subsequent inhibition of growth, may be measured in vivo by methods commonly known in the art.

In addition to the in vitro and in vivo methods, certain methods of the invention may be performed ex vivo in a subject, to manipulate one or more cell types within the subject. An “ex vivo” method, as used herein, is a method, which involves isolation of a cell from a subject, manipulation of the cell outside of the body, and reimplantation of the manipulated cell into the subject.

For a local anesthetic spinosyn formulation to be effective, the effect must be achievable at the desired site. For an antiarrhythmic spinosyn formulation to be effective, the effect must be achievable at myocardium. The pharmacological absorption and distribution of the spinosyn or spinosyn formulation and its bioavailability, will influence the dose, route and frequency of administration of the drug in order to achieve an effective dose at the desired site. “Bioavailability” is the degree to which the pharmaceutically active agent becomes available to the target tissue after the agent's introduction into the body. Enhancement of the bioavailability of a pharmaceutically active agent can provide a more efficient and effective treatment for patients because, for a given dose, more of the pharmaceutically active agent will be available at the targeted tissue sites.

Pharmaceutically effective amount and administration routes for the spinosyn compositions according to the present invention, may be optionally selected by those skilled in the art depending on, the location we need the anesthetic effect, or the desired duration of the effect and the species into which the anesthetic or antiarrhythmic is administered. More specifically, dosage and concentrations will change depending on the size of nerve, species, anatomic location (peripheral nerve, epidural space, intrathecal), and even the volume of injectate, the severity of a patient's arrhythmia, the simultaneous use of a pacemaker, the therapeutic strategy, and the age, weight, sex, general health conditions and racial (genetic) background of a patient.

The spinosyn local anesthetic or antiarrhythmic compositions according to the present invention are administered by any art recognized method including topically, enterally or parenterally in an amount which is effective for treating the specific indication. The topical route of administration includes for example, inhalational, intranasal, intravaginal, cutaneous, intravitreal, and transdermal. The enteral administration involves oral administration including sublingual and rectal. Parenteral administration includes, intravenous, intradermal, subcutaneous, intramuscular, interperitoneal, intraartirial, intracerebral, intracardiac, intraosseous, intrathecal, intravesical route. Parenteral administration also includes continuous infusion e.g., during 2 hours or 12 hours or even 24 hours so as to achieve better distribution of the drug to the target site and better bioavailability.

The spinosyn compositions may be administered to the host, in a fast state, together with food or after meals.

The spinosyn pharmaceutical composition according to the present invention may be prepared into any formulation known by those skilled in the art appropriate for the chosen administration route. For oral administration the compounds can be formulated into solid or liquid preparations, for example, tablets, lozenges capsules, powders, solutions, emulsions, suspensions and dispersions, colloidal dispersion systems including nanocapsules and microspheres. Such preparations are well known in the art, as are other oral dosage regimes not listed here. The formulation may also be administered topically to skin or mucous membranes as an ointment, lotion, emulsion, solution cream, powder, oil, gel, foam or spray. For application by the ophthalmic mucous membrane route, compositions of the present invention may be formulated as eye drops in a physiologically acceptable diluent such as water, saline or DMSO or eye ointments or gels in conventional ocular preparations. Exemplary materials that may be suitable for inclusion in ophthalmic formulations are hydrogels, carbopols, polyacrylic acids, cellosic viscosity enhancing materials and chitosan. Important properties may include adherence to the mucin coat and the corneal surface of the eye to increase residence time of the composition. Cyclodextrins may also be employed in ophthalmic formulations to increase the solubility of the actives in solution. Hyalauronic acid may also be included to increase precorneal residence time. The spinosyns of the present invention may be also formulated for aerosol administration, particularly to the respiratory tract, intranasal administration and skin topical application.

Pharmaceutical compositions of this invention suitable for parenteral administration comprise a composition of spinosyn or spinosyn derivative of salt thereof, in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Administration can also be by transmucosal, cutaneous or transdermal means. The composition can be, for example, in form of an ointment, gel, lotion, cream, oil, emulsion, paste, suspension, powder or spray-solution, foam or aerosol. The composition can be incorporated into a bandage or plaster. For transmucosal or transdermal administration, penetrants appropriate to the barrier to be permeated are used in the formulation. In one embodiment, the transdermal delivery agent can be, for example, DMSO, urea, 1-methyl-2-pyrrolidone, oleic acid, or a terpene (e.g., 1-menthol, d-limonene, RS-(+/−)-beta-citronellol, geraniol). Transdermal delivery systems can include, e.g., patches which deliver a pharmacon continuously through unbroken skin for periods of hours to days to weeks, depending on the particular patch. For transmucosal administration, penetrants appropriate to the barrier to be permeated are used in the formulation, for facilitating the delivery of the drug. Such penetrants are generally known in the art. Exemplified transdermal delivery formulations that can find use in the present invention include those described in U.S. Pat. Nos. 6,589,549; 6,544,548; 6,517,864; 6,512,010; 6,465,006; 6,379,696; 6,312,717 and 6,310,177, each of which are incorporated herein by reference.

Other delivery systems suitable for use with the present invention include time-release, delayed release, sustained release, or controlled release delivery systems. Such systems may avoid repeated administrations in many cases, increasing convenience to the animal or human and the physician. Many types of release delivery systems are available and known to those of ordinary skill in the art.

Implantation may be used according to one embodiment of the present invention and includes inserting implantable drug delivery systems, e.g., microspheres, hydrogels, polymeric systems and non-polymeric systems into a host. Use of a long-term release implant may be particularly suitable in some embodiments of the invention. “Long-term release,” as used herein, means that the implant containing the composition is constructed and arranged to deliver therapeutically effective levels of the composition for at least 15 or 45 days, and preferably at least 60 or 90 days, or even longer in some cases. Long-term release implants are well known to those of ordinary skill in the art.

Proteins which are water insoluble, such as zein, can also be used as materials for the formation of spinosyn containing microparticles. Additionally, proteins, polysaccharides and combinations thereof which are water soluble can be formulated with spinosyn into microparticles and subsequently cross-linked to form an insoluble network. For example, cyclodextrins can be complexed with individual drug molecules and subsequently cross-linked. Encapsulation or incorporation of spinosyn into carrier materials to produce drug containing microparticles can be achieved through known pharmaceutical formulation techniques. In the case of formulation in fats, waxes or wax-like materials, the carrier material is typically heated above its melting temperature and the drug is added to form a mixture comprising drug particles suspended in the carrier material, drug dissolved in the carrier material, or a mixture thereof. Microparticles can be subsequently formulated through several methods including, but not limited to, the processes of congealing, extrusion, spray chilling or aqueous dispersion. For some carrier materials it may be desirable to use a solvent evaporation technique to produce drug containing microparticles. In this case drug and carrier material are co-dissolved in a mutual solvent and microparticles can subsequently be produced by several techniques including, but not limited to, forming an emulsion in water or other appropriate media, spray drying or by evaporating off the solvent from the bulk solution and milling the resulting material.

In some embodiments, the spinosyn in a particulate form is homogeneously dispersed in a water-insoluble or slowly water soluble material. To minimize the size of the drug particles within the composition, the drug powder itself may be milled to generate fine particles prior to formulation. The process of jet milling, known in the pharmaceutical art, can be used for this purpose.

For systemic administration, it may be useful to encapsulate the composition in nanoparticles (nanospheres) or liposomes. Nanoparticles exploit biological pathways to achieve payload delivery of molecules to cellular and intracellular targets. Synthetic strategies, including surface, porosity, stealthing and size modifications can be utilized to refine the pharmacokinetic properties of nanoparticles and allow for efficient delivery. The average diameter of such nanoparticles in a composition can range for example from 1-1000 nm. Liposomes are artificial membrane vessels which are useful as a delivery vector in vivo or in vitro and in which a variety of drugs can be incorporated. It has been shown that large unilamellar vessels (LUV), which range in size from 0.2 micrometers to 4.0 micrometers can encapsulate large macromolecules within the aqueous interior and these macromolecules can be delivered to cells in a biologically active form (Fraley, et al., Trends BioChem. Sci., 6:77, 1981). Liposomes for example are encapsulated in a polymer matrix, the liposomes being sensitive to specific stimuli, e.g., temperature, pH, light or a degrading enzyme. Entrapment of drugs into liposomes constitutes an attractive approach to improve and facilitate the delivery of active agents to cells and to reduce toxic effects associated with their administration. Because of the similarity of the primary components of liposomes with natural membranes, liposomes are generally non-toxic and biodegradable. In addition liposomes could protect drugs against enzymatic degradation, improve their pharmacokinetics and tissue distribution and may allow a controlled release of therapeutic agents to appropriate cells. In addition, the distribution and therapeutic availability of liposomes can be modulated through variations of their size, lamellarity, lipid composition, charge and surface properties.

We may also have systems in which the composition is encapsulated by an ionically-coated microcapsule with a microcapsule core-degrading enzyme. Examples of systems in which release of the drug is gradual and continuous include, e.g., erosional systems in which the composition is contained in a form within a matrix and effusional systems in which the composition permeates at a controlled rate, e.g., through a polymer. Such sustained release systems can be e.g., in the form of pellets, or capsules.

Finally the spinosyn of the present invention may be conjugated to a water soluble polymer, such as, polyglutamic acid, or a polyaspartic acid or albumin protein or to a water soluble metal chelator, in a way that can achieve higher water solubility than the unconjugated drug.

Any method or process can be used to prepare the pharmaceutical compositions of the invention, known by the skilled artisan. Solid dosage forms can be prepared by wet granulation, dry granulation, direct compression and the like. The solid dosage forms of the present invention may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer, which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

During the preparation of the pharmaceutical compositions according to the present invention the spinosyn is usually mixed with a carrier or excipient, which may be a solid, semi-solid, or liquid material with processes and methods known in the art.

The parenteral formulations can be presented in unit-dose or multi-dose sealed containers, such as ampoules and vials, and can be stored in a freeze-dried (lyophilized) condition requiring only the addition of the sterile liquid carrier, for example, water, for injections, immediately prior to use. They can also be sold in separate vials which are mixed by the practitioner just prior to use, in order to avoid stability problems of the final formulation.

Spinosyn or a derivative or salt thereof may be also administered as a prodrug. The term “prodrug” as used herein means a pharmacologically acceptable derivative of the compound, such that an in vivo biotransformation of the derivative gives the active compound as a result of i.e., spontaneous chemical reaction(s), enzyme catalyzed chemical reaction(s), photolysis, and/or metabolic chemical reaction(s). A prodrug is thus a covalently modified analog or latent form of a therapeutically-active compound.

“Prodrug moiety” refers to a labile functional group which separates from the active compound during metabolism, systemically, inside a cell, by hydrolysis, enzymatic cleavage, or by some other process (Bundgaard, Hans, “Design and Application of Prodrugs” in A Textbook of Drug Design and Development (1991), P. Krogsgaard-Larsen and H. Bundgaard, Eds. Harwood Academic Publishers, pp. 113-191). Enzymes which are capable of an enzymatic activation mechanism with the spinosyn prodrug compounds of the invention include, but are not limited to, transaminases, cytochrome CYP450, oxidoreductases, epimerases, dehydratases, methyl transferases, amidases, esterases, phospholipases, cholinesterases, etc. A prodrug moiety may include an active metabolite or the drug itself.

Prodrugs of spinosyns may be prepared by modifying functional groups present in the spinosyns in such a way that the modifications are cleaved in vivo to give the parent spinosyn. Prodrugs are often useful because, in some situations they may be easier to administer than the parent drug. They may, for instance, be bioavailable by oral administration whereas the parent drug is not.

Dosage may be adjusted appropriately to achieve desired drug levels, local or systemic, depending upon the mode of administration. The doses may be given in one or several administrations per day. In most cases the spinosyn dosage for humans and animals will be from 0.05 μg/kg body weight to about 2000 mg/kg body weight daily, administered in one or multiple doses, specifically from 1 mg/kg to about 1000 mg/kg daily or from 5 mg/kg to 200 mg/kg daily and even better from 10 mg/kg to 45 mg/kg, or 10 mg/kg to 50 mg/kg, daily. In the specific case of topical administration dosages are preferably administered from about 0.05 μg to 500 mg spinosyns per cm² or better from about 10 μg to 50 mg per cm² or even better from 0.1 mg to 10 mg per cm². Parental administration, in some cases, may be from one to several orders of magnitude lower dose per day, as compared to oral doses.

The term “carrier” as used herein, denotes any organic or inorganic ingredient, which may be natural or synthetic, with which one or more spinosyns of the invention are combined to facilitate their administration to the host in need thereof. The carrier may be co-mingled or otherwise mixed with one or more spinosyns of the present invention, in a manner such that we can get the desired pharmaceutical efficacy. The carrier may be either soluble or insoluble, depending on the application. Carriers include pharmaceutically-acceptable carriers, solvents, excipients, diluents, permeation enhancers, buffering agents, isotonifiers, preservatives, adjuvants, including immunologic adjuvants, for example, squalene, stabilizers, ionic and non-ionic surfactants or detergents or emulsifiers for example, sodium deoxycholate, which are nontoxic to humans and animals exposed thereto at the dosages and concentrations employed. Carriers are used as formulation ingredients, for example, to stabilize or protect the active ingredient within the composition before use, to facilitate the drug delivery, etc. Carriers, as used herein, include also any molecule, for example, a protein, e.g., albumin, which can create conjugates with the spinosyn and thus, facilitates the spinosyn-conjugate (i.e., serum albumin-spinosad conjugate) delivery to the target cells. Carriers further include food components and any art recognized diluent or composition that could be used for the administration of spinosyn to the target. Still further, the food carriers include fish and animal feed compositions containing carbohydrates, fats, vitamins, proteins and the like. Non-limiting examples of various physiologically-acceptable carriers may be chosen from an aqueous pH buffered solution, dimethyl sulfoxide (DMSO), oleic acid, alcohols, for example, ethyl alcohol, cetyl alcohol, ketones, ethers, esters and the like, oil and fats such as olive oil, peanut oil, castor oil, corn oil, wheat germ oil, cotton seed oil, silica, cellulose and cellulose derivatives, silicones and siloxanes, hydrophobic and other polymers, calcium stearate, calcium laurate, zinc chloride, magnesium chloride, sodium chloride, sodium lactate, sodium metabisulfite, magnesium oleate, cyclodextrins, mineral oil, white petrolatum, emulsifying wax, pectin, starch, talc, lecithin, proteins, for example, human or bovine serum albumin and others known in the art.

The terms “pharmaceutically-acceptable carrier” or “physiologically-acceptable carrier” as used herein, includes those carriers that are, within the scope of sound medical judgment, suitable for use in contact with the tissues of humans and animals without undue toxicity, irritation, and allergic response, and are commensurate with a reasonable benefit/risk ratio. Pharmaceutically-acceptable carriers are well known in the art.

The term “penetration or permeation enhancers” includes compounds that have been evaluated for penetration enhancing activity, including sulphoxides (e.g., dimethylsulfoxide (DMSO) and decylmethylsulfoxide (CIOMSO)), Azones (e.g., laurocapram), pyrrolidones (for example 2-pyrrolidone, 2P), alcohols and alkanols (ethanol, or decanol), glycols (for example, propylene glycol is a common excipient in topically applied dosage forms), terpenes (e.g. a-terpinol) and 4-tert-butyl cyclohexanol (TBCH), sulfoxide decylmethylsulfoxide (CioMSO); ethers such as diethylene glycol monoethyl ether, dekaoxyethylene-oleylether, and diethylene glycol monomethyl ethers; surfactants such as sodium lauryl sulfate (SLS), sodium octyl sulfate (SOS), dodecyltriethylammonium bromide (DDAB), octyltriethylammonium bromide (OTAB), TWEEN® 20 and TWEEN® 80, fatty acids such as C₈-C₂₂ and other fatty acids, C₈-C₂₂ fatty alcohols, and polyols.

Buffering agents help to maintain the pH in the range which approximates physiological conditions. Suitable buffering agents for use with the present invention include both organic and inorganic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium glyuconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additional possibilities include phosphate buffers, histidine buffers and trimethylamine salts.

Preservatives may be added to retard microbial growth. Suitable preservatives for use with the present invention include phenol, benzyl alcohol, meta-cresol, methyl paraben, propyl paraben, octadecyldimethylbenzyl ammonium chloride, benzalkonium halides (e.g., benzalkonium chloride, bromide or iodide), hexamethonium chloride, alkyl parabens such as methyl or propyl paraben, catechol, resorcinol, cyclohexanol and 3-pentanol, formaldehyde, metals for example, mercury or metal salts and combinations thereof.

Isotonicifiers may be added to ensure isotonicity of liquid compositions and include polyhydric sugar alcohols, preferably trihydric or higher sugar alcohols, such as glycerin, erythritol, arabitol, xylitol, sorbitol and mannitol.

Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol, propylene glycol, polyethylene glycol 400; amino acid polymers; sulfur-containing reducing agents, such as urea, glutathione, thioctic acid, sodium thioglycolate, thioglycerol, alpha-monothioglycerol and sodium thiosulfate; low molecular weight polypeptides (i.e., <10 residues); proteins such as human serum albumin, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran.

Non-ionic surfactants or detergents (also known as “wetting agents”) may be present to help solubilize the therapeutic agent especially in the case of molecules with very low water solubility, like the spinosyn molecule. Suitable non-ionic surfactants include polysorbates (20, 80, etc.), polyoxamers (184, 188, etc.), Pluronic® polyols, and polyoxyethylene sorbitan monoethers (Tween®-20, Tween®-80, etc.).

Additional miscellaneous excipients may include bulking agents or fillers (e.g., starch), chelating agents (e.g., EDTA), antioxidants (e.g., ascorbic acid, methionine, vitamin E) and cosolvents, formulation detectable markers, aerosol propellants, sunscreen agents, perfumes or essential oils, powder base such as for example kaolin and a skin conditioning agent.

A skin conditioning agent, as defined herein, improves dry or damaged skin. Such agents, for example, include: acetyl cysteine, N-acetyl dihydrosphingosine, acrylates/behenyl acrylate/dimethicone acrylate copolymer, adenosine, adenosine cyclic phosphate, adenosine phosphate, adenosine triphosphate, alanine, albumen, algae extract, allantoin and derivatives, aloe barbadensis extracts, aluminum PCA, amyloglucosidase, arbutin, arginine, azulene, bromelain, buttermilk powder, butylene glycol, caffeine, calcium gluconate, capsaicin, carbocysteine, carnosine, beta-carotene, casein, catalase, cephalins, ceramides, chamomilla recutita (matricaria) flower extract, cholecalciferol, cholesteryl esters, coco-betaine, coenzyme A, corn starch, cycloethoxymethicone, cysteine DNA, cytochrome C, darutoside, dextran sulfate, dimethicone copolyols, dimethylsilanol hyaluronate, elastin, elastin amino acids, epidermal growth factor, ergocalciferol, ergosterol, ethylhexyl PCA, fibronectin, folic acid, gelatin, gliadin, beta-glucan, glucose, glycine, glycogen, glycolipids, glycoproteins, glycosaminoglycans, glycosphingolipids, horseradish peroxidase, hydrogenated proteins, hydrolyzed proteins, jojoba oil, keratin, keratin amino acids, kinetin, lactoferrin, lanosterol, lauryl PCA, lecithin, linoleic acid, linolenic acid, lipase, lysine, lysozyme, malt extract, maltodextrin, melanin, methionine, mineral salts, niacin, niacinamide, oat amino acids, oryzanol, palmitoyl hydrolyzed proteins, pancreatin, papain, PEG, pepsin, phospholipids, phytosterols, placental enzymes, placental lipids, pyridoxal 5-phosphate, quercetin, resorcinol acetate, riboflavin, RNA, saccharomyces lysate extract, silk amino acids, sorbitol, sphingolipids, stearamidopropyl betaine, stearyl palmitate, tocopherol, tocopheryl acetate, tocopheryl linoleate, ubiquinone, vitis vinifera (grape) seed oil, wheat amino acids, xanthan gum, and zinc gluconate.

In addition to the active or therapeutic ingredients, the pharmaceutical compositions according to the present invention may contain a number of inert materials referred to herein as excipients. These materials help to impart satisfactory processing and compression characteristics to the formulation including diluents, binders, glidants and lubricants. A further group of excipients helps to give additional desirable physical characteristics to the finished product. Included in this group are disintegrants, colors, flavors and sweetening agents, polymers or waxes or other solubility-retarding materials.

The primary active ingredient for use in the present invention comprises at least one spinosyn from the class of spinosyns as described above. The percentage of spinosyn in the pharmaceutical composition required for effective local anesthetic effect and/or antiarrhythmic effect, may vary substantially depending on the spinosyn itself, the desired intensity of the effect, the method of administration, other additives or actives present in the composition that may influence the effectiveness of spinosyn, the condition of patient or host to be treated, such as his immunologic response and clinical image, age, body weight, sex, sensitivity, food, time of administration, medicine to be administered concurrently, degree of arrhythmia of the patient, etc. The appropriate dose and times of administration of the spinosyn under certain conditions, may be decided through preliminary tests for determining an optimal dose, by a medical specialist in account of the above-mentioned guideline. The upper limit of concentration may be also driven by characteristics of toxicity that would be readily apparent to the skilled artisan.

The spinosyns of the present invention may be also administered together with one or more of antipyretics, analgesics, antiemetics and antiallergy drugs.

The invention also discloses a method of providing local anesthetic effect and pain relief and also a method of treating arrhythmia, by administering to a host, a formulation containing, an effective amount of spinosyn or spinosyn derivative or salt thereof. Optionally an additional therapeutic agent may be administered with spinosyn.

One skilled in the art would recognize that the amount of spinosyn could be reduced in the event one or more additional active agents were present, for example, other local anesthetics and/or antiarrhythmic agents, so long as the combined composition is effective as a local anesthetic or antiarrhythmic agent. The spinosyns of the present invention may be used with one, two (triple) or even three (quadruple) other agents in a multi-drug combination therapy and the agents may be contained in the same formulation or in different formulations. An additional agent may, for example, be selected from the group consisting of biocides, ectoparasites, natural substances (like geraniol oil), enzyme inhibitors, for example, kinase inhibitors, biomolecule mimics, analytes (including a nanoparticle, an environmental contaminant, or a toxin), antiviral agents, sedative agents, like for example medetomidine, anticancer agents, antibiotics, antibacterials, antimetabolites, polypeptides, corticosteroids, antidepressants, immunomodulatory agents, antibodies, cytokines, antiprotozoan, agents used for the parasitic infection diseases, vasoconstrictors like for example adrenaline, vasodilators, emollients such as for example acetyl arginine, anticoagulants, haemostatic agents, analgesic anti-inflammatory agents, other local anesthetics, other antiarrhythmic agents such as for example quinidine, propafenone, ambasilide, amiodarone, dronedarone, ibutilide, azimilide, sematilide, tedisamile, disopyramide, flecainide, sotalol, bretylium, dofetilide, almokalant, bepridil, clofilium, clotrimazole, ketoconazole, bupivacaine, erythromycin, verapamil, nifedipine, zatebradine, bisindolylmaleimide; other cardiovascular agents such as, but not limited to, ACE inhibitors such as benazepril, captopril, enalapril, fosinopril, lisinopril, moexipril, perindopril erbumine, quinapril, ramipril, and trandolapril; angiotensin II antagonists such as candesartan, eprosartan, irbesartan, losartan, olmesartan, telmisartan, and valsartan; cardiac glycosides such as digoxin, L-type calcium channel blockers, T-type calcium channel blockers, selective and nonselective beta blockers, endothelin antagonists, thrombin inhibitors, warfarin, factor Xa inhibitors, low molecular weight heparin, unfractionated heparin, clopidogrel, ticlopidine, Ilb/Illa receptor antagonists such as tirofiban, 5HT receptor antagonists, integrin receptor antagonists, thromboxane receptor antagonists, TAFI inhibitors and P2T receptor antagonists. Compounds of the invention can also be administered as the sole active ingredient. Furthermore compounds of the invention may be administered in combination with a pacemaker or defibrillator device.

The term “natural substance” as used herein, includes any chemical compound or substance or product, produced by a living organism found in nature, that usually has a pharmacological or biological activity and may be used as a drug or drug synergist. A natural product can be considered as such even if it can be prepared by synthetic means. Natural substances may be from plant origin, for example, extracts from terrestrial plan tissues, from marine organisms from example, from corals, sponges etc, from animal sources for example, some venoms, microbial metabolites resulting from microorganism fermentation broths, etc.

When additional active ingredients are used in combination with the spinosyn compositions of the present invention, the spinosyn composition and the at least one additional active ingredient can be administered to the host simultaneously, sequentially or separately. If the administration is not simultaneous, the compounds may be administered in a close time proximity to each other or after long intervals. Furthermore, in the case of simultaneous administration, it does not matter if the compounds are administered in the same dosage form (composition) or in different compositions or even by different administration routes, e.g., one compound may be administered topically or intravenously and another compound may be administered orally. In addition, the different components of the combination can, independently of the other, follow different dosing schedules, e.g., the spinosyn compounds may be administered daily for a week, or every second week for a three months period, while the at least one additional active is administered one time or twice per week for three weeks followed by a one week period wherein the compound is not administered.

The spinosyn compositions of the invention can be administered once-daily, twice-daily, three times daily, as an instant dose or by continuous infusion (i.e., 1h or 2h infusion), once-weekly or once-monthly or in any other dosage protocol. In addition, the administration can be continuous, i.e., every day, or intermittently. The terms “intermittent” or “intermittently” as used herein means stopping and starting at either regular or irregular intervals. For example, intermittent administration can be administration one to six days per week or it may mean administration in cycles (e.g., daily administration for two to eight consecutive weeks, then a rest period with no administration for up to one week) or it may mean administration on alternate days.

According to one embodiment, the spinosyn is present in the composition in an amount in the range from 0.01 μg/ml or grams of composition to 999 mg/ml or grams of composition. According to another embodiment, the spinosyn is present in an amount in the range from 1 μg/ml or grams of composition to 200 mg/ml or grams of composition. According to yet another embodiment, the spinosyn is present in an amount in the range from 10 μg/ml or grams of composition to 100 mg/ml or grams of composition.

According to another embodiment, spinosyn compositions may contain from about 0.001% to about 99.9% by weight of spinosyn, and preferably from about 0.1% to about 50% by weight and most preferably from 1% to 10% by weight.

The content or percentage of the composition that is spinosyn may vary widely depending on the drug form (capsule, liquid, liposome, etc.), the route of administration, the need for further dilution or the addition of a carrier prior to use (injectables), etc. Therefore the formulation/product's concentration of spinosyn is only informative and used in the calculation and adjustment of the proper spinosyn dose expressed as mg/kg/day or mg/m²/day or mg/cm² per application, to the host. Selection of a dosage is within the skill of an ordinary artisan, and may be accomplished by starting at a relatively low dosage and increasing the dosage until an acceptable effect is achieved.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches. Notwithstanding that the numerical ranges and parameters setting forth the broad scope of the invention are approximations, unless otherwise indicated the numerical values set forth in the specific examples are reported as precisely as possible, based on the average findings of replicates carried out. Any numerical value, however, inherently contain certain errors necessarily resulting from the standard deviation found in their respective testing measurements.

The following examples are provided to describe the invention in further detail. These examples are intended merely to illustrate specific embodiments of the compositions and methods of the invention, and should in no way be construed as limiting the invention. These examples provide the results of tests conducted to determine a) the in vivo local anesthetic effect of spinosyns and b) the in vitro inhibition of hERG channels which is indicative of an antiarrhythmic effect. It will be apparent to those skilled in the art that embodiments described herein may be modified or revised in various ways without departing from the spirit and scope of the invention.

### Local Anesthetic Tests

### EXAMPLE 1

### In Vivo Test on Rats-Comparison with Lidocaine

Test sample: spinosad (Supplier: Sigma-Aldrich/Fluka) in DMSO 9 mg/ml (0.8% w/w or approx. 12 mM)

Positive control: Lidocaine (Manufacturer: VetTek), as a Lidocaine Hydrochloride (2% solution or approx. 74 mM)

Control: DMSO

Assay: Pain response testing-foot pinch

Animals used: male Sprague-Dawley rats

Experimental set-up. The test article was administered as follows:

TABLE 1
Experimental set-up

| | | | | | ENTARCO | | | |
|---|---|---|---|---|---|---|---|---|
| | Number | | Control | Lidocaine | Test Article | | | |
| Group | of | Animal | Vehicle | (Positive | spinosad | Dose | Volume | Study |
| No. | Animals | Numbers | (DMSO) | Control) | (mg/kg) | Route | (mL/kg) | Day |
| 1 | 6M | 1001-1006 | * | NA | NA | SQ | 1.33 | 1 |
| 2 | 6M | 2001-2006 | NA | * | NA | SQ | 1.33 | 1 |
| 3 | 6M | 3001-3006 | NA | NA | 12 | SQ | 1.33 | 1 |

M = Male
SQ = Subcutaneous

Approximately 1.33 mL/kg spinosad solution was administered once, subcutaneously at 12 mg/kg (9 mg/mL, 1.33 mL/kg) into the left hind foot of each rat in the treatment group (Group 3), positive control group (Group 2, Lidocaine, 1.33 mL/kg), and control vehicle group (Group 1, DMSO, 1.33 mL/kg).

Pain Response Testing

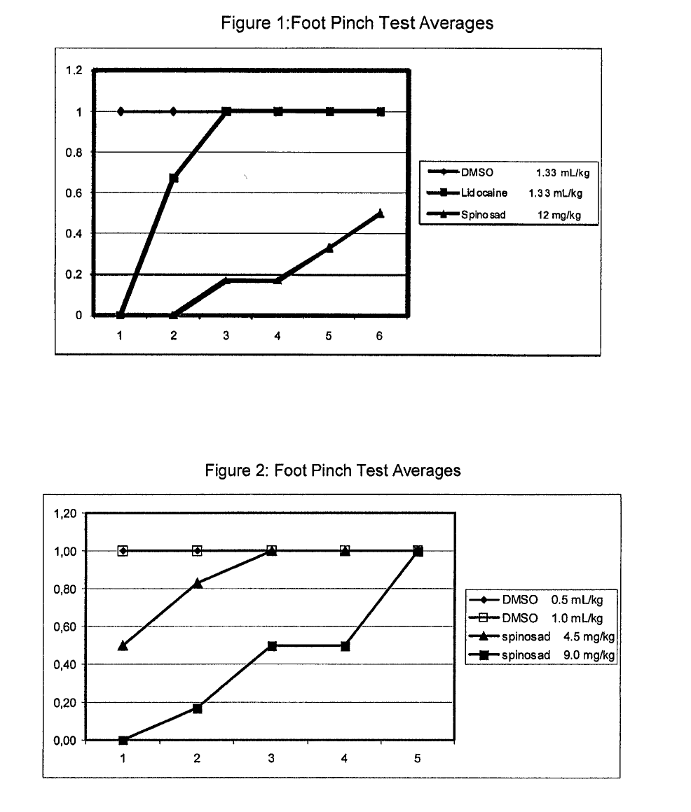

Beginning approximately 45 minutes post-dose all animals were subjected to pain response testing, foot pinch test. The test was conducted on each rat, in order, and then repeated on each, 5 more times (6 rounds of testing) through 4 hours post-dose. The foot pinch test measured the rodent's tolerance of pressure on their foot. The rat was removed from its cage and the treated foot was pinched. Whether or not the rodent's foot responded to the pinch was documented. The treatment group averages were compared to positive and negative control group averages (Table 3 and FIG. 1).

Six total rounds of foot pinch-pain response testing were conducted at approximately 45, 70, 90, 120, 150, and 240 minutes post-dose. Time of dosing and pain testing results were recorded for each animal.

Pain Response Testing Results:

TABLE 2
Foot Pinch Test Results

| Animal Number | Round 1 | Round 2 | Round 3 | Round 4 | Round 5 | Round 6 |
|---|---|---|---|---|---|---|
| 1001 | 1.00 | 1.00 | 1.00 | 1.00 | 1.00 | 1.00 |
| 1002 | 1.00 | 1.00 | 1.00 | 1.00 | 1.00 | 1.00 |
| 1003 | 1.00 | 1.00 | 1.00 | 1.00 | 1.00 | 1.00 |
| 1004 | 1.00 | 1.00 | 1.00 | 1.00 | 1.00 | 1.00 |
| 1005 | 1.00 | 1.00 | 1.00 | 1.00 | 1.00 | 1.00 |
| 1006 | 1.00 | 1.00 | 1.00 | 1.00 | 1.00 | 1.00 |
| Average per animal | 1.00 | 1.00 | 1.00 | 1.00 | 1.00 | 1.00 |
| 2001 | 0.00 | 1.00 | 1.00 | 1.00 | 1.00 | 1.00 |
| 2002 | 0.00 | 1.00 | 1.00 | 1.00 | 1.00 | 1.00 |
| 2003 | 0.00 | 1.00 | 1.00 | 1.00 | 1.00 | 1.00 |
| 2004 | 0.00 | 1.00 | 1.00 | 1.00 | 1.00 | 1.00 |
| 2005 | 0.00 | 0.00 | 1.00 | 1.00 | 1.00 | 1.00 |
| 2006 | 0.00 | 0.00 | 1.00 | 1.00 | 1.00 | 1.00 |
| Average per animal | 0.00 | 0.67 | 1.00 | 1.00 | 1.00 | 1.00 |
| 3001 | 0.00 | 0.00 | 0.00 | 0.00 | 0.00 | 0.00 |
| 3002 | 0.00 | 0.00 | 0.00 | 0.00 | 0.00 | 0.00 |
| 3003 | 0.00 | 0.00 | 0.00 | 0.00 | 0.00 | 0.00 |
| 3004 | 0.00 | 0.00 | 1.00 | 1.00 | 1.00 | 1.00 |
| 3005 | 0.00 | 0.00 | 0.00 | 0.00 | 1.00 | 1.00 |
| 3006 | 0.00 | 0.00 | 0.00 | 0.00 | 0.00 | 1.00 |
| Average per animal | 0.00 | 0.00 | 0.17 | 0.17 | 0.33 | 0.50 |

1 = No Anesthetic effect
0 = Anesthetic effect present
Round 1 = 45 minutes post dose
Round 2 = 70 minutes post dose
Round 3 = 1.5 hour post dose
Round 4 = 2 hours post dose
Round 5 = 2.5 hours post dose
Round 6 = 4 hours post dose

TABLE 3
Foot Pinch Test Averages

| Group | Dose mg/kg/day | | round 1 | round 2 | round 3 | round 4 | round 5 | round 6 |
|---|---|---|---|---|---|---|---|---|
| 1 | DMSO | average | 1.00 | 1.00 | 1.00 | 1.00 | 1.00 | 1.00 |
| | 1.33 mL/kg | | | | | | | |
| 2 | Lidocaine | average | 0.00 | 0.67 | 1.00 | 1.00 | 1.00 | 1.00 |
| | 1.33 mL/kg | | | | | | | |
| | (26.6 mg/kg) | | | | | | | |
| 3 | Spinosad | average | 0.00 | 0.00 | 0.17 | 0.17 | 0.33 | 0.50 |
| | 1.33 mL/kg | | | | | | | |
| | (12 mg/kg) | | | | | | | |

Conclusion: Based on the live-phase results of this study, it is concluded that a single subcutaneous spinosad dose into the footpad at 12 mg/kg was well tolerated by male Sprague-Dawley rats and resulted in a local anesthetic effect that remained unresolved over the duration of this study (4 hours). The Lidocaine group showed a localized effect similar to spinosad but which resolved by round 2 (˜70 minutes post-dose) of foot pinch pain response testing, while the vehicle-only group (DMSO) had no anesthetic effect through the duration of the study. It has to be noted here, that the amount of lidocaine used (expressed in mM) was remarkably higher in comparison with spinosad, thus demonstrating the superior anesthetic effect of spinosad on a molecular basis. Moreover spinosad has a much higher LD₅₀ than lidocaine, suggesting spinosad's much lower toxicity.

The above data show that spinosyns, and spinosad in particular, have considerable long lasting local anesthetic properties and thus, can induce a prolonged local anesthetic effect.

### EXAMPLE 2

### In Vivo Test on Rats-Duration of Action and Reversible Effects

This test was conducted to establish spinosad's reversible local anesthetic effects and confirm the prolonged effect. The same assay as in example 1 was used.

Test article: spinosad in DMSO 9 mg/ml, administered in two doses; high dose 9 mg/kg and low dose 4.5 mg/kg.

Control: DMSO

The treatment group averages were compared to positive and negative control group averages (Table 6 and FIG. 2).

TABLE 4
Experimental set-up

| | | | Solvent | Dose | | |
|---|---|---|---|---|---|---|
| | Number | | (Vehicle) or | Volume | | |
| Group | of | Animal | 1312 | (foot pad) | Dose | Study |
| Number | Animals | Numbers | Formulation | mL/kg | Route | Day |
| 1 | 2M | 1001-1002 | DMSO | 0.5 | SQ | 1 |
| 2 | 3M | 2001-2003 | DMSO | 1.00 | SQ | 1 |
| 3 | 6M | 3001-3006 | spinosad Low | 0.5 | SQ | 1 |
| | | | Dose | | | |
| | | | 4.5 mg/kg | | | |
| 4 | 6M | 4001-4006 | spinosad | 1.00 | SQ | 1 |
| | | | High Dose | | | |
| | | | 9.0 mg/kg | | | |

M = Males
SQ = Subcutaneous

TABLE 5
Foot Pinch Test Results

| Animal | | | | | |
|---|---|---|---|---|---|
| Number | Round 1 | Round 2 | Round 3 | Round 4 | Round 5 |
| 1001 | 1.00 | 1.00 | 1.00 | 1.00 | 1.00 |
| 1002 | 1.00 | 1.00 | 1.00 | 1.00 | 1.00 |
| Average per | 1.00 | 1.00 | 1.00 | 1.00 | 1.00 |
| animal | | | | | |
| 2001 | 1.00 | 1.00 | 1.00 | 1.00 | 1.00 |
| 2002 | 1.00 | 1.00 | 1.00 | 1.00 | 1.00 |
| 2003 | 1.00 | 1.00 | 1.00 | 1.00 | 1.00 |
| Average per | 1.00 | 1.00 | 1.00 | 1.00 | 1.00 |
| animal | | | | | |
| 3001 | 1.00 | 1.00 | 1.00 | 1.00 | 1.00 |
| 3002 | 0.00 | 1.00 | 1.00 | 1.00 | 1.00 |
| 3003 | 1.00 | 1.00 | 1.00 | 1.00 | 1.00 |
| 3004 | 1.00 | 1.00 | 1.00 | 1.00 | 1.00 |
| 3005 | 0.00 | 0.00 | 1.00 | 1.00 | 1.00 |
| 3006 | 0.00 | 1.00 | 1.00 | 1.00 | 1.00 |
| Average per | 0.50 | 0.83 | 1.00 | 1.00 | 1.00 |
| animal | | | | | |
| 4001 | 0.00 | 0.00 | 0.00 | 0.00 | 1.00 |
| 4002 | 0.00 | 0.00 | 0.00 | 0.00 | 1.00 |
| 4003 | 0.00 | 0.00 | 0.00 | 0.00 | 1.00 |
| 4004 | 0.00 | 1.00 | 1.00 | 1.00 | 1.00 |
| 4005 | 0.00 | 0.00 | 1.00 | 1.00 | 1.00 |
| 4006 | 0.00 | 0.00 | 1.00 | 1.00 | 1.00 |
| Average per | 0.00 | 0.17 | 0.50 | 0.50 | 1.00 |
| animal | | | | | |

1 = No Anesthetic effect
0 = Anesthetic effect present
Round 1 = 1 hour post dose
Round 2 = 4 hours post dose
Round 3 = 6 hours post dose
Round 4 = 8 hours post dose
Round 5 = 24 hours post dose

TABLE 6
Foot Pinch Test Averages

| | | | | round | | round |
|---|---|---|---|---|---|---|
| Group | Dose mg/kg/day | round 1 | round 2 | 3 | round 4 | 5 |
| 1 | DMSO 0.5 mL/kg | 1.00 | 1.00 | 1.00 | 1.00 | 1.00 |
| 2 | DMSO 1.0 mL/kg | 1.00 | 1.00 | 1.00 | 1.00 | 1.00 |
| 3 | spinosad 4.5 mg/kg | 0.50 | 0.83 | 1.00 | 1.00 | 1.00 |
| 4 | spinosad 9.0 mg/kg | 0.00 | 0.17 | 0.50 | 0.50 | 1.00 |

Conclusion: Based on the live-phase results of this study, it is concluded that a single subcutaneous spinosad dose into the footpad at 9 mg/kg was well tolerated by male Sprague-Dawley rats and resulted in a local anesthetic effect that remained unresolved over a duration of at least 6-8 hours, but it was fully reversible at 24 hours. The lower dose of 4.5 mg/kg had a shorter local anesthetic effect, which lasted during the first hour of study only. The vehicle-only group (DMSO) had no anesthetic effect through the duration of the study.

The above data show that spinosyns, and spinosad in particular, have reversible local anesthetic properties, the duration of which is dose related.

### Antiarrhythmic Tests

### EXAMPLE 3

### Effects of Spinosad on the Cloned hERG Potassium Channels

Rational for Ion channel selection: Inhibition of hERG (I_{Kr}) channels closely predicts the cardiac action potential prolongation. HERG potassium channels are expressed in a human embryonic kidney (HEK293) cell line which does not have any endogenous I_{Kr}. This stable expressed hERG cell line is widely used for hERG testing. The percentage inhibition of spinosad on human-ERG currents were evaluated from the gold standard manual patch clamp studies.

Test sample: spinosad (Supplier:Sigma-Aldrich/Fluka), in DMSO 9 mg/ml (0.8% w/w or approx. 12 mM)

Positive control: Terfenadine (Supplier: Sigma-Aldrich), in DMSO at a concentration of 1 mM.

Control: Previous studies showed that 0.3% DMSO did not affect hERG currents. All the final solutions contained no more than 0.3% DMSO.

Assay: Electrophysiology recording: Micropipette was pulled from borosilicate glass with the pipette tip resistance between 3-5 MΩ. For each experiment, a single dish of cells is removed from the incubator, washed twice with room temperature ECS and then placed on the microscope stage. A commercial patch clamp amplifier was used for the whole cell recordings.

Voltage protocol and current recording: The tail currents were evoked at room temperature once every 30 s by a 3 s −50 mV repolarizing pulse following a 2 s+50 mV depolarizing pulse with a hold voltage of −80 mV. A 50 ms depolarized pulse to −50 mV at the beginning of the voltage protocol served as a baseline for calculating the amplitude of the peak tail current. Only stable cells with recording parameters above threshold were allowed to enter the drug application procedure. The hERG currents were allowed to stabilize over a 3 minutes period in the presence of a vehicle alone prior to the application of spinosad. The cells were kept in the test solution until the peak tail current was stable (<5% change) for about 5 sweeps or for a maximum of 6 min, whichever came first.

Acceptance criteria for measurements: 1. Whole-cell membrane resistance (Rm) was greater than 1000 MΩ throughout the experiment. 2. Initial current was greater than 300 pA. 3. Series resistance value following establishment of whole-cell mode was less than 12 MΩ. 4. The leak current that the cell began with or developed over time was less than 10% of the ionic current magnitude (up to a maximum of 80 pA).

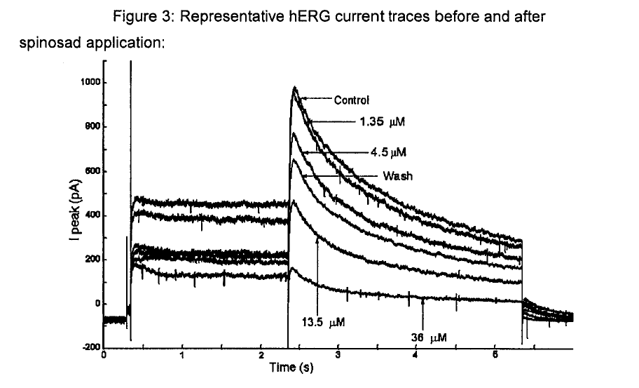

Results: 1.35 μM, 4.5 μM, 13.5 μM and 36 μM spinosad solution were tested for their effects on hERG currents and inhibited currents by 7.2±8.9%, 21.8±4.4%, 41.7±8.4% and 78.3±5.5% respectively. Table 7 below provides the percent inhibition of the individual data point and the statistics of the percent inhibition of different spinosad concentrations on hERG currents. The representative hERG voltage-clamp current recordings acquired at the steady state during control and after application of spinosad solution are superimposed in FIG. 3. Spinosad blocking on hERG current was reversible after one minute of saline wash. The positive control Terfenadine inhibited hERG current with an IC₅₀ at 43 nM, This IC₅₀ was consistent with the laboratory historical data.

TABLE 7
Percentage inhibition of hERG currents by spinosad

| | | % | % | % | % | | |
|---|---|---|---|---|---|---|---|
| Test | Concentration | Block | Block | Block | Block | Average | |
| Article ID | (μM) | Cell #1 | Cell #2 | Cell #3 | Cell #4 | Inhibition (%) | SD |
| | 1.35 | 17.3 | 3.5 | 0.6 | | 7.2 | 8.9 |
| spinosad | 4.50 | 26.9 | 19.2 | 19.4 | | 21.8 | 4.4 |
| | 13.50 | 30.3 | 48.4 | 47.7 | 40.2 | 41.7 | 8.4 |
| | 36.00 | | 77.5 | 84.1 | 73.2 | 78.3 | 5.5 |

Conclusion: Spinosad, when tested for hERG activity using whole cell recordings from a HEK293-hERG stable cell line, inhibited hERG currents in a concentration-dependent manner.

Thus, using the methodology described above, it has been shown that spinosad exhibits activity in the hERG assay (inhibition of hERG channels), thereby confirming the utility of spinosyns as antiarrhythmics.

1. A method of treating a host in need of a local anesthetic agent, comprising administering in said host an effective amount of a composition containing at least one spinosyn or salt thereof and a suitable carrier, wherein the at least one spinosyn or salt thereof is the only local anesthetic agent in said composition.

2. The method of claim 1, wherein the condition in need of a local anesthetic agent is at least one of, acute pain, chronic pain, myofascial pain, muscle pain, back pain, postherpetic neuralgia, osteoarthritis, osteomyelitis, venipuncture, hair removal, topical surgical operation, dental operation, operations on the musculoskeletal system, spinal/epidural anesthesia and pruritus.

3. The method of claim 1, wherein the spinosyn is chosen from at least one or more of spinosyn A, spinosyn D, spinosad, spinetoram, butenyl spinosyn.

4. The method of claim 1, wherein the spinosyn is chosen from at least one or more of spinosyn A and spinosyn D.

5. The method of claim 1, wherein the spinosyn is administered to the host in an amount of 0.05 μg/kg body weight daily to 2000 mg/kg body weight daily, once or in multiple doses or by continuous infusion.

6. The method of claim 1, wherein the spinosyn is administered to the host in an amount of 1 mg/kg body weight daily to 100 mg/kg body weight daily, once or in multiple doses or by continuous infusion.

7. The method of claim 1, wherein the spinosyn is administered topically to the host in a dosage from about 0.05 μg to 100 mg spinosyns per cm2.

8. The method of claim 1, wherein at least one or more additional active agent is administered to the host, simultaneously, contained in the same spinosyn composition or in another composition, separately or sequentially.

9. The method of claim 8, wherein the at least one additional active agent is chosen from at least one or more of biocides, ectoparasites, natural substances, enzyme inhibitors, antiviral agents, fungicidal agents, sedative agents, anticancer agents, antibiotics, antibacterials, corticosteroids, antiprotozoan agent, vasoconstrictors, emollients, anticoagulants, haemostatic agents, analgesic or anti-inflammatory agents.

10. The method of claim 8, wherein the at least one additional active agent is another local anesthetic agent, a sedative or a general anesthesia agent.

11. The method of claim 8, wherein the at least one additional active agent is chosen from at least one or more of bupivacaine, proparacaine, benoxinate, lidocaine, tolycaine, tocainide, articaine, benzocaine, etidocaine, pramoxine, dyclonine, benzyl alcohol, menthol, eugenol, phenol, cocaine, medetomidine, mepivacaine, tetracaine, procaine, propoxycaine, meprylcaine, amethocaine, prilocaine, levobupivacaine, dibucaine and ropivacaine, including their derivatives, analogs, isomers and salts thereof.

12. The method of claim 1 wherein the local anesthetic effect must have a long duration of action.

13. The method of claim 1 wherein the local anesthetic effect must last until at least 24h.

14. The method of claim 1, wherein the spinosyn composition when formulated accordingly and injected for local pain relief, demonstrates performance of full sensory response returning in 3-5 days.

15. The method of claim 1, wherein the composition comprising at least one spinosyn or salt thereof as an anesthetic agent, is in the form of one or more of a solution, dispersion, ointment, gel, lotion, cream, oil, emulsion, paste, suspension, spray-foam or aerosol, solution or powder for injection, patch, implantable delivery system, capsule, tablet, pill, lozenge, hydrogel, dry powder, colloidal dispersion system, microparticle, nanoparticle or liposome.

16. The method of claim 1, wherein the host is a human.

17. The method of claim 1, wherein the host is an animal.

18. The method of claim 1, wherein the composition is administered to the host enterally, parenterally or topically.

19. A method of treating a host, in need of an antiarrhythmic agent, comprising administering to said host an effective amount of a composition comprising at least one spinosyn or spinosyn or salt thereof as an antiarrhythmic agent and a suitable carrier.

20. The method of claim 19 wherein the composition comprising at least one spinosyn or spinosyn or salt thereof as an antiarrhythmic agent, is in the form of one or more of a solution, dispersion, ointment, gel, lotion, cream, oil, emulsion, paste, suspension, spray-foam or aerosol, solution or powder for injection, patch, implantable delivery system, capsule, tablet, pill, lozenge, hydrogel, dry powder, colloidal dispersion system, microparticle, nanoparticle or liposome.

21. The method of claim 19, wherein the spinosyn is chosen from at least one or more of spinosyn A, spinosyn D, spinosad, spinetoram, butenyl spinosyn.

22. The method of claim 19, wherein the spinosyn is chosen from at least one or more of spinosyn A and spinosyn D.

23. The method of claim 19, wherein the spinosyn is administered to the host in an amount of 0.05 μg/kg body weight daily to 2000 mg/kg body weight daily once or in multiple doses or by continuous infusion.

24. The method of claim 19, wherein the spinosyn is administered to the host in an amount of 1 mg/kg body weight daily to 100 mg/kg body weight daily once or in multiple doses or by continuous infusion.

25. The method of claim 19, wherein at least one additional active agent is administered to the host simultaneously, contained in the same spinosyn composition or in another composition, separately or sequentially.

26. The method of claim 19, wherein the at least one additional active agent is chosen from at least one or more of biocides, ectoparasites, natural substances, enzyme inhibitors, antiviral agents, anticancer agents, antibiotics, antibacterials, corticosteroids, antiprotozoan agent, vasoconstrictors, emollients, anticoagulants, haemostatic agents, other antiarrhythmic agents, analgesic or anti-inflammatory agents, ACE inhibitors, angiotensin II antagonists, cardiac glycosides, L-type calcium channel blockers, T-type calcium channel blockers, selective and nonselective beta blockers, endothelin antagonists, thrombin inhibitors, aspirin, nonselective NSAIDs, warfarin, factor Xa inhibitors, low molecular weight heparin, unfractionated heparin, clopidogrel, ticlopidine, lib/lila receptor antagonists, 5HT receptor antagonists, integrin receptor antagonists, thromboxane receptor antagonists, TAFI inhibitors and P2T receptor antagonists.

27. The method of claim 19, wherein the host is a human or an animal.

28. The method of claim 19, wherein the composition is administered to the host enterally, parenterally or topically.

29. The method of claim 19, wherein the condition according to which the host is in need of an antiarrhythmic agent, is atrial fibrillation.

30. The method of claim 19, wherein the condition according to which the host is in need of an antiarrhythmic agent, is selected from the group consisting of atrial flutter, atrial arrhythmia and supraventricular tachycardia.

31. The method of claim 19, wherein an antitachycardia device is used in combination with the spinosyn containing composition.
